(19) United States
(12) Patent Application Publication
(10) Pub. No.: US 2011/0253140 A1
(43) Pub. Date: Oct. 20, 2011

(54) FORMULATIONS CONTAINING LARGE-SIZE CARRIER PARTICLES FOR DRY POWDER INHALATION AEROSOLS

(75) Inventors: Hugh D.C. Smyth, West Lake Hills, TX (US); Martin Donovan, Austin, TX (US)

(73) Assignee: STC.UNM, Albuquerque, NM (US)

(21) Appl. No.: 13/056,588

(22) PCT Filed: Jul. 30, 2009
(86) PCT No.: PCT/US09/52277

§ 371 Date: Jul. 5, 2011

Related U.S. Application Data
(60) Provisional application No. 61/084,805, filed on Jul. 30, 2008.

(51) Int. Cl. A61M 15/00; A61P 11/00; A61K 9/16

(52) U.S. Cl. 128/203.15; 424/490; 428/402

(57) ABSTRACT

A dry powder inhaler may include a drug chamber configured to contain a formulation including carrier particles and working agent particles, a mouthpiece configured to direct flow of working agent particles to a user, and a retaining member proximal the mouthpiece. The retaining member be sized and arranged to prevent flow of substantially all carrier particles to the user while permitting flow of working agent particles to a user. The inhaler may include a formulation including carrier particles for delivering working agent to the pulmonary system of a patient. The carrier particles may have an average sieve diameter greater than about 500 μm. The carrier particles may be one of polystyrene, PTFE, silicone glass, and silica gel or glass.

### CROSS-REFERENCE TO RELATED APPLICATIONS

This application is related to U.S. provisional patent application No. 61/084,805, entitled “FORMULATIONS CONTAINING LARGE-SIZE CARRIER PARTICLES FOR DRY POWDER INHALATION AEROSOLS,” filed on Jul. 30, 2008, which is incorporated herein by reference.

### TECHNICAL FIELD

The present invention is directed generally to dry powder inhalation aerosols and methods of delivering drug and/or therapeutic agents to a patient. More particularly, the present invention is directed to formulations containing large-size carrier particles for dry powder inhalation aerosols and methods of delivering the same to a patient.

### BACKGROUND

The benefits of inhaled therapy for treatment of lung diseases such as asthma, chronic obstructive pulmonary disease (COPD), and cystic fibrosis have been recognized for many years. Direct administration of drug to the airways minimizes systemic side effects, provides maximum pulmonary specificity, and imparts a rapid duration of action.

Dry powder inhalers (DPIs) are becoming a leading device for delivery of therapeutics to the airways of patients. Currently, all marketed dry powder inhalation products are comprised of micronized drug (either agglomerated or blended) delivered from “passive” dry powder inhalers, DPIs. These inhalers are passive in the sense that they rely on the patient's inspiratory effort to disperse the powder into a respirable aerosol.

Despite their popularity and the pharmaceutical advantages over other inhaler types, passive dry powder inhalers typically have relatively poor performance with regard to consistency. In particular, DPIs emit different doses depending on how the patient uses the device, for example, the inhalation effort of the patient.

Also, the efficiency of DPIs can be quite poor. In one study comparing the performance of the two most widely prescribed DPIs, only between 6% and 21% of the dose emitted from the device was considered respirable. Improved performance for DPI devices is desperately needed from both a clinical and product development standpoint. One promising approach to improving DPI performance is to modify the formulation rather than the device itself.

Conventional formulations for dry powder inhalation aerosols typically contain micronized drug of particle sizes small enough to enter the airways and be deposited in the lung. To make these highly cohesive and very fine particles dispersible, so called “carrier” particles are mixed with the drug particles. These carrier particles are found in nearly all dry powder inhaler products currently marketed. The carrier particles serve to increase the fluidization of the drug because the drug particles are normally too small to be influenced significantly by the airflow through the inhaler. The carrier particles thus improve the dose uniformity by acting as a diluent in the formulation.

Although these carrier particles, which are generally about 50-100 microns in size, improve the performance of dry powder aerosols, the performance of dry powder aerosols remains relatively poor. For instance, only approximately 30% of the drug in a typical dry powder aerosol formulation will be delivered to the target site, and often much less. Significant amounts of drug are not released from these conventional carrier particles and, due to the relatively large size of the carrier in relation to the drug, the drug is deposited in the throat and mouth of the patient where it may exert unwanted side effects. The dogma in the field is that carrier particle sizes greater than about 100 microns lead to poorer performance.

A dry powder formulation is typically a binary mixture, consisting of micronized drug particles (<5 μm) and larger inert carrier particles (typically lactose monohydrate with 63-90 μm diameters). Drug particles experience cohesive forces with other drug particles and adhesive forces with carrier particles (predominately Van der Waals forces), and it is these interparticulate forces that must be overcome in order to effectively disperse the powder and increase lung deposition efficiency. The energy used to overcome the interparticulate forces is provided by the inspired breath of the patient as they use the inhaler. The aerodynamic forces entrain and deaggregate the powder, though variations in the inhalation effort of the patient (e.g. such as those arising from fibrosis or obstruction of the airways) significantly affect the dispersion and deposition of the drug, producing the flow-rate dependency of the inhaler. Obviously, there is a need for improved dry powder formulations employing novel carrier particles to maximize the safety and efficacy profiles of current DPI inhalers.

The active pharmaceutical ingredient (API) typically constitutes less than 5% of the formulation (w/w), with lactose comprising the vast majority of the dose. The purpose of the carrier lactose is to prevent aggregation of the drug particles due to cohesive forces, primarily Van der Waals forces arising from the instantaneous dipole moments between neighboring drug particles. Due to the small size of the drug particles these resulting cohesive forces are quite strong and not readily broken apart by the aerodynamic force provided by inhalation, producing aggregates that possess poor flow properties and end up depositing in the back of the throat. By employing a binary mixture, the drug adheres to the carriers particles instead and the larger size of the carrier particles allows them to be more easily entrained in the air stream produced when the patient inhales, carrying the API toward a mesh where the carrier particle collides; the force from the collision is often sufficient to detach the drug particles from the carrier, dispersing them in the airstream and allowing their deposition within the lung. However, a large fraction of API remains attached to carriers that do not collide effectively with the mesh, but instead are deflected, producing insufficient force to disperse the drug particles from its surface. API that does not dissociate from these carriers, along with drug adhered to carrier particles that slip through without any contact with the mesh, are deposited in the back of the throat via inertial impaction, often causing significant side effects in the throat.

Carrier particle interactions have been investigated by several researchers. For drug carrier formulations, the detachment of the drug from the carrier particle surface is determined by the drag forces experienced in the inhaled air stream, the cohesive forces between drug particles, and the adhesive forces between the drug and carrier. Therefore, any means of increasing the relative effects of the drag forces, such as increasing the air velocity within the inhaler, will result in more drug particles detaching from the carrier particle surface, resulting in higher lung deposition efficiencies. Kassem (1990) showed that even after extremely high flow rates however, significant amounts of drug are still found adhered to the carrier particles.

As shown in FIG. 1, interparticulate or adhesional forces keep the particles in the static state and aerodynamic forces help the particles to fluidize and then deaggregate. In other words, fine powders (<5 μm) generate fine aerosols, but particle adhesion reduces delivery efficiency and leads to flow rate dependent lung deposition. For example, one published study found that lung deposition for the corticosteroid, budesonide, was 27.7% of the metered dose at a peak inspiratory flow rate (PIF) of 60 L/min, but only 14.8% at a PIF of 35 L/min. While this may be acceptable for drugs with a large therapeutic index like budesonide, it may not be acceptable for drugs with a narrow therapeutic index such as, for example, proteins and peptides. Hence, it may be advantageous to develop powder formulations with improved dispersibility from passive DPIs.

Referring to FIGS. 1A and 1B, the mechanisms of powder dispersion for dry powder inhalers is shown. FIG. 1A illustrates the static powder held together by the interparticulate forces which are overcome by the aerodynamic forces to produce fluidization and deaggregation. FIG. 1B depicts the same event at the level of the particles with the large carrier particles attached to small drug particles going from an aggregated state to a dispersed state. As illustrated, changing carrier particle density and size may affect respirable dose. In FIG. 2, the relationships between adhesive forces (interparticulate) and dispersion forces (aerodynamic), as calculated for idealized systems, are plotted.

Carrier particles have been used for approximately thirty (30) years, and many studies looking at various properties of the carrier particles have been performed and reported in the scientific literature. Several studies have investigated the use of different sizes of carrier particles to improve the performance of dry powder inhaler formulations. For example, Islam et al. (2004) reported the influence of carrier particle size on drug dispersion of salmeterol xinafoate. According to Islam et al., the particle size of the lactose carrier in the mixtures was varied using a range of commercial inhalation-grade lactoses. The dispersion of the drug appeared to increase as the particle size of the lactose carrier decreased.

The effect of carrier size on drug dispersion has been reported by others:
Bell J H, Hartley P S, Cox J S G. 1971. Dry powder aerosols. I. A new powder inhalation device. J Pharm Sci 60(10):1559-1564.
Ganderton D. 1992. The generation of respirable clouds from coarse powder aggregate. J Biopharm Sci 3(1/2):101-105.
French D L, Edward D A, Niven R W. 1996. The influence of formulation on emission, deaggregation, and deposition of dry powders for inhalation. J Aerosol Sci 27(5):769-783.
Kassem N M, Ho K K L, Ganderton D. 1989. The effect of air flow and carrier size on the characteristics of an inspirable cloud. J Pharm Pharmacol 41:14 P.
Steckel H, Muller B W. 1997. In vitro evaluation of dry powder inhalers. II. Influence of carrier particle size and concentration on in vitro deposition. Int J Pharm 154:31-37.

For example, the greatest dispersion of cromolyn sodium from an interactive dry powder inhaler mixture at a flow rate of 60 L/min was observed with lactose particles sized between 70-100 microns (Bell et al. 1971). Using binary mixtures of salbutamol sulfate and a sugar carrier, the FPF decreased with increasing carrier particle size in the studies of Stricana et al (1998). A reduction in carrier size improved respirable fraction of albuterol sulfate (Ganderton 1992; Kassem et al. 1989) and budesonide (Steckel, Muller 1997). However, a higher respirable fraction of terbutaline sulfate was obtained from coarser lactose (53-105 microns) than from a finer lactose (<53 microns) (Byron et al. 1990). Therefore the literature relating to conventional dry powder inhaler formulations teaches us that a carrier particle size less than around 100 microns is preferable, but the carrier particle size should be greater than approximately 50 microns.

These findings are recognized in the patent literature. For example, in U.S. Pat. No. 6,153,224, what is claimed is a powder for use in a dry powder inhaler, the powder comprising active particles and carrier particles for carrying the active particles. The powder contains additive material on the surfaces of the carrier particles to promote the release of the active particles from the carrier particles during inhalation. It is important to note that these inventors define the particle size of the carrier particles to have a diameter which lies between 20 microns and 1000 microns but 95% of the additive material is in the form of particles having a diameter of less than 150 microns. Additionally, this patent specifies that the carrier particles comprise one or more crystalline sugars such as an a lactose monohydrate.

In U.S. Pat. No. 5,376,386, the average size of carrier is preferably in the range 5 to 1000 microns, and more preferably in the range 30 to 250 microns, and most preferably 50 to 100 microns. The carrier is a crystalline non-toxic material having a rugosity of less than 1.75. The preferred carriers are monosaccharides, disaccharides, and polysaccharides. In U.S. Pat. No. 7,090,870, a pharmaceutical excipient useful in the formulation of dry powder inhaler compositions comprises a particulate roller-dried anhydrous β-lactose, with the β-lactose particles having a size between 50 and 250 micrometers and a rugosity between 1.9 and 2.4.

There are many reviews on the influence of formulation on DPI performance, and in most cases these have focused on modifications to carrier particles in terms of size, surface rugosity, crystallinity, moisture content, and other parameters. Although carriers appear apt targets for tuning inhaler performance there are several problems with this approach. First, the parameters that can be changed for carriers are inter-related, so manipulating one parameter usually produces corresponding alterations in others. For example, attempts to manipulate surface properties of lactose carriers, e.g. milled vs. spray-dried, are often accompanied by significant changes in particle size, surface area, powder density, etc. This has, so far, precluded systematic and well-controlled studies of how these parameters can be modulated to influence performance. Secondly, the design window available remains small because formulators are restricted to one or two excipient materials in which these properties can only be varied within small magnitudes. Furthermore, there is currently little evidence that these parameters can be tuned to properties of the drug or characteristics of the inhaler. A greater understanding of carrier particle design control is critical not only for tunability but also to understand current formulation variability.

Some conventional DPIs permit, and sometimes even intend, carrier particles to exit the inhaler. As a result, in the United States, the FDA restricts the carrier particle material to lactose. There may be a need for advanced formulation technologies including alternative carrier particle materials that may be more judiciously chosen based on hygroscopic properties of the carrier (e.g., a dessicant material) and the surface interactions (e.g., acid or base character of the drug and carrier) between the carrier and the drug. Thus, it may be desirable to provide a DPI that retains substantially all carrier particles to allow for circumventing the FDA restriction of lactose as the carrier material.

This disclosure may solve one or more of the aforesaid problems via therapeutic formulations containing large-size carrier particles, significantly greater than 100 microns, for dry powder inhalation aerosols and methods of delivering the same to a patient. These much larger carrier particles will have improved performance at sizes larger than has been studied or published before. There may be other advantages to this approach also. For example, according to some aspects, because the novel carrier particles have much larger sizes, they can be captured in the DPI device and never need to enter the patient. This may allow the use of many different materials that would not necessarily be amenable for delivery to a patient, and thus could not previously be used in conventional DPIs.

### SUMMARY OF INVENTION

In accordance with various aspects, the present disclosure is directed to a dry powder inhaler comprising a drug chamber configured to contain a formulation including carrier particles and working agent particles, a mouthpiece configured to direct flow of working agent particles to a user, and a retaining member proximal the mouthpiece. The retaining member be sized and arranged to prevent flow of substantially all carrier particles to the user while permitting flow of working agent particles to a user.

In some aspects, the retaining member may be configured to prevent flow of carrier particles having a sieve diameter greater than about 250 microns while permitting flow of working agent particles having a sieve diameter less than about 250 microns. In some aspects, the retaining member may be configured to prevent flow of carrier particles having a sieve diameter greater than about 500 microns while permitting flow of working agent particles having a sieve diameter less than about 500 microns.

According to various aspects, the inhaler may include a formulation including carrier particles for delivering working agent to the pulmonary system of a patient via a dry powder inhaler. In some aspects, the carrier particles may have an average sieve diameter greater than about 500 μm, or greater than about 1000 μm, or about 5000 μm. According to various aspects, the formulation further comprises particles of working agent adhered to the carrier particles.

In some aspects, the carrier particles may comprise one of polystyrene, polytetrafluoroethylene (PTFE, aka Teflon), silicone glass, and silica gel or glass. In some aspects, the carrier particles may comprise biodegradable material.

According to some aspects of the disclosure, a formulation for a dry powder inhaler may comprise carrier particles for delivering working agent to the pulmonary system of a patient via a dry powder inhaler. The carrier particles may comprise one of polystyrene, PTFE, silicone glass, and silica gel or glass and may have an average sieve diameter greater than about 500 μm. The formulation may further comprise particles of working agent adhered to the carrier particles. In various aspects, the carrier particles may have an average sieve diameter greater than about 1000 μm. The carrier particles may have an average sieve diameter of about 5000 μm.

In accordance with various aspects of the disclosure, a formulation for a dry powder inhaler may comprise carrier particles for delivering working agent to the pulmonary system of a patient via a dry powder inhaler. The carrier particles may have an average sieve diameter greater than about 1000 μm. For example, the carrier particles may have a sieve diameter of about 5000 μm. The formulation may further comprise particles of working agent adhered to the carrier particles. The carrier particles may comprise biodegradable material or nonbiodegradable material. The nonbiodegradable material may comprise polystyrene, PTFE, silicone glass, or silica gel or glass.

### BRIEF DESCRIPTION OF THE DRAWINGS

FIGS. 1A and 1B are schematic illustrations of the mechanisms of powder dispersion for dry powder inhalers.

FIG. 2 is a graph showing the influence of carrier particle size on the relative forces of adhesion and aerodynamic dispersion.

FIGS. 3A-3C are diagrammatic illustrations of an exemplary dry powder inhaler in accordance with various aspects of the disclosure.

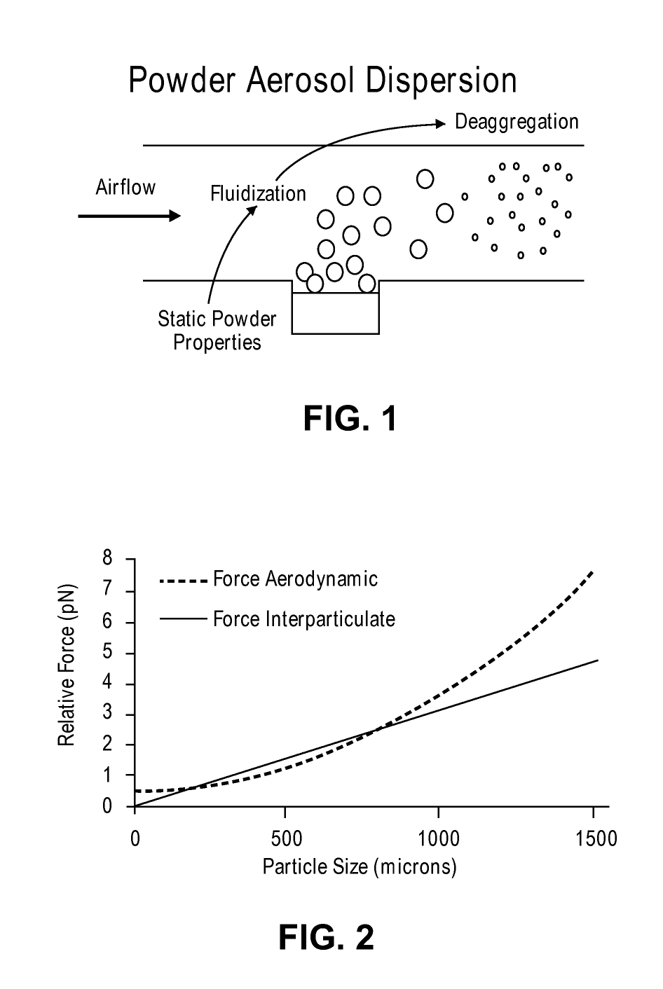

### DETAILED DESCRIPTION

Exemplary embodiments of formulations that improve the performance of dry powder inhalation aerosols for the delivery of therapeutic agents to the airways of patients are described herein. Exemplary carrier particles are disclosed for improved delivery of therapeutic and other working agents to the respiratory tract. Carrier particles in accordance with this disclosure are orders of magnitude larger than those used in current inhaler formulations. The working agents that can be delivered via the particles include, but are not limited to a therapeutic agent, diagnostic agent, prophylactic agent, imaging agent, or combinations thereof.

It will be understood that the term “working agent” includes material which is biologically active, in the sense that it is able to increase or decrease the rate of a process in a biological environment. The working agent referred to throughout this disclosure may be material of one or a mixture of pharmaceutical product(s).

Large carrier particles (>1 mm) of various materials can be used to improve and possibly tune DPI performance. Order of magnitude calculations for adhesion forces and aerodynamic detachment forces indicate that aerodynamic forces exceed adhesion forces not only when carrier particles have diameters less than approximately 100 microns (current DPI formulations rely on this approach) but also when the diameters are greater than around 700 microns (FIG. 2). In the case where these carrier particles are large, a dry powder inhaler device may include a retaining member designed to retain them (e.g. using a mesh), circumventing concerns about the toxicity of the carrier material.

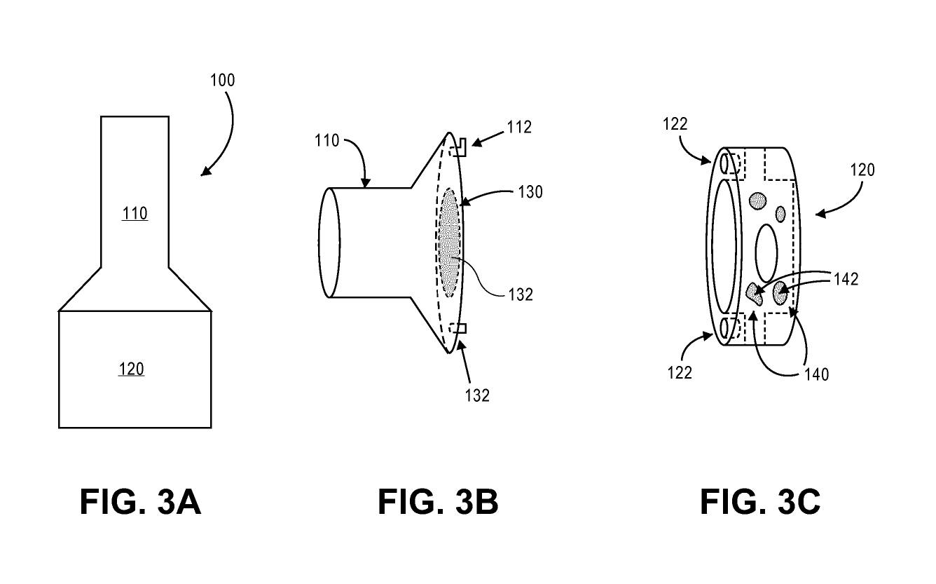

Referring now to FIGS. 3A-3C, an exemplary dry powder inhaler 100 is shown. The dry powder inhaler 100 may include a mouthpiece 110 and a drug chamber 120. The mouthpiece 110 and the drug chamber 120 may be coupled together by coupling members 112 and complementary openings 122 sized and arranged for receiving the coupling members 112. Alternatively, the mouthpiece 110 and the drug chamber 120 may be coupled together in any known matter or may be integrally formed as a single piece construction. The drug chamber 120 may include an opening 124 configured to receive a capsule (not shown) containing the carrier particles 140 with working agent 142 adhered thereto. The drug chamber 120 may also include a mechanism (not shown) structured and arranged to open the capsule and disperse the carrier particles with working agent. One skilled in the art would appreciate the myriad of conventional capsules and mechanisms for opening, all of which are contemplated by this disclosure.

One or more retaining members 130 having openings 132 may be at or near the mouthpiece 110, at the interface of the mouthpiece 110 and the drug chamber 120, or at an end of the drug chamber 120 near the mouthpiece 110. According to various aspects, the one or more retaining members 130 may comprise a mesh, a screen, orifices, channels, nozzles, or the like. Regardless of its/their structure, the one or more retaining members 130 are sized and arranged to prevent substantially all carrier particles 140 from exiting the inhaler 100 while permitting working agent particles 142 to exit the inhaler 100.

According to various aspects of the disclosure, the carrier particles 140 are large enough in any two dimensions relative to the openings 132 in the retaining members 130 such that the carrier particles 140 are prevented from exiting the inhaler 100 through the mouthpiece 110. For example, the carrier particles 140 may have a sieve diameter greater than about 500 microns. In some aspects, the average sieve diameter may be greater than about 1000 microns (1 mm). In some aspects, the average sieve diameter may be greater than about 5000 microns (5 mm).

Despite their large sizes, the carrier particles 140 in accordance with the disclosure are capable of achieving high de-aggregation forces within the inhaler that effectively disperse the drug. For these large carrier particles, effective dispersion is achieved when the carrier particle collides with the one or more retaining members 130 located near the mouthpiece 110 of the inhaler 100, and the force imparted to the working agent particle is strong enough to overcome the adhesive forces between the carrier and the working agent. This impaction/dispersive force results from the change in momentum that occurs when the moving carrier particle collides with the retaining member 130, and is given by

$${F}_{impaction} = \begin{matrix}{mt}_{∞}^{′} \\ {t}_{C}\end{matrix} \qquad ( 1 )$$

where m is the mass of the carrier particle, t_{c} is the collision time (the length of time that the carrier particle is in contact mesh; on the order of ˜10 μs), and v_{∞} is the velocity of the air stream (22).

The velocity of the airstream is given by:

$${v}_{∞} = \frac{Q}{A} \qquad ( 2 )$$

where Q is the inspiratory flow rate in L min⁻¹, and A is the cross sectional area of the dry powder inhaler.

One thing to note is that the mass of the particle, and consequently the dispersive force, is proportional to the cube of the carrier particle diameter. This is in contrast with the adhesive force, which has only a linear dependence on the diameter of the carrier particle. The adhesive force preventing the effective entrainment and dispersion of the powder is given by:

$${F}_{adhesion} = \frac{{A}_{H} {d}_{1} {d}_{2}}{12 {D}^{2} ( {d}_{1} + {d}_{2} )} \qquad ( 3 )$$

where A_{H} is the Hamaker's constant, and is typically on the range of 10⁻¹⁹ J, D is the interparticulate distance and is commonly given as 4 Angstroms (10⁻¹⁰ m), and d₁ and d₂ are the diameters of the drug and carrier particles respectively (23).

However, as the inertia of the particles increases with size, these large particles must be made from low density materials, such as polystyrene, so that they will be effectively entrained in the flow stream. In vitro dispersion studies in our lab have shown that compared to standard lactose carrier particles, formulations using polystyrene beads (diameter 1.41-2.36 mm) exhibit increased fine particle fractions, coupled to a greater degree of flow rate independence. This technology has the potential to use a wide range of carrier particle materials to optimize drug-carrier interactions (i.e. changing surface chemistry, surface roughness, particle density, etc) with much greater freedom than current carrier systems allow.

Carrier particles in accordance with the disclosure may permit quantification of the respirable dose and flow rate dependency of, for example, a model asthma drug intended to be delivered to the lung as an aerosol using an array of novel carrier particles. Large carrier particles in accordance with the disclosure, for example, carrier particles greater than about 500 μm in diameter, and in some aspects greater than about 1000 μm, will have improved emitted dose efficiency, improved respirable dose efficiency, and less flow rate dependency than conventional dry powder formulations available in currently marketed products.

According to various aspects of the present disclosure, applicant has surprisingly and unexpectedly found that larger carrier particle sizes, for example in excess of 500 μm in diameter, and in some aspects greater than 1000 μm, and in some aspects 4000-5000 μm or greater than 5000 μm, may be preferable over the conventionally-sized carrier particles.

Morphology of carrier particles has been shown to have a significant influence on the performance of the dry powder inhaler system (Zeng et al., 1998, 1999, 2000a, 2000b). It has been postulated that batch-to-batch variability of lactose carrier performance in dry powder inhaler systems can be attributed to differences in carrier particle shape and morphology resulting from changes in crystallization environment (Zeng, Martin, Marriott, 2001).

In accordance with various aspects of the disclosure, larger carrier particles greater than 500 microns or greater than 1000 microns or greater than 5000 microns can be generated in many different shapes. For example, instead of spherical or crystalline shapes, any regular or irregular shaped flake or bead, including discs, polygons, doughnut-shapes, flat plates, or squares can be prepared to increase respirable fractions. The shape of the carrier particles can be controlled by using technologies such as, for example, milling, spray drying, extrusion, polymer imprinting, and others. Although the term “bead” may be used throughout this disclosure in referring to the carrier particles, it should be appreciated that the carrier particles may comprise any of the aforementioned shapes.

According to various aspects, surface smoothness or rugosity of carrier particles can have some influence on the performance of the dry powder inhaler formulation. By changing the materials of the carrier particles, rather than being restricted by the use and modification of sugar particles, different carrier particle smoothness levels can be more easily achieved. In some aspects, coatings may be applied to the surface of the carrier particles. Since the carrier particles are not inhaled and do not leave the inhaler device, the carrier particles may be made of many different materials, including materials that would be potentially toxic if included in devices and formulations that are currently used. For example, the carrier particles may include a polystyrene coating. Polystyrene is not biodegradable and therefore should not enter the patient's airways. According to various aspects, the use of biodegradable and non-biodegradable carriers and biodegradable and non-biodegradable coatings on carriers may be facilitated by retaining the carrier particles in the device upon actuation and patient inhalation. This retention in the device is made possible by the larger sizes of the carrier particles, for example, greater than 500 microns or greater than 1000 microns or greater than 5000 microns, that provide better respirable fractions.

Persons skilled in the art would appreciate that the density of the drug particles is important for the performance of dry powder inhaler formulations (Edwards et al). Again, inhalation of the carrier particles can be prevented by increasing their particle size to very large particles, for example, greater than 500 microns or greater than 1000 microns or greater than 5000 microns. Due to retention of the larger particles by an inhalation device, the carrier particle composition may be selected from many different materials. For example, glass beads have a much higher density than lactose beads. Polystyrene beads can have much lower density than lactose beads. There are many different materials that could be chosen to have the optimal particle density for a particular inhaler design, for a particular drug, and/or for a particular patient with a specific breathing capacity. Therefore, according to various aspects of the disclosure, the range of carrier particle densities can be selected to optimize the inhaler performance without being restricted to the densities of sugars like lactose, sucrose, mannitol, and other inert materials currently used in dry powder inhalers.

The powder flow of the carrier powders is important to the formulation of dry powder inhalers because the uniformity of filling individual doses (i.e. the variability of dose weight measured out) can be correlated with powder flowability. This is important for prepackage cavity doses, such as, for example, capsules, blister strip cavities, etc., as well as for devices that sample powder from an internal reservoir. Increased flowability may lead to higher uniformity of powder dosing, which may improve dry powder inhaler performance.

Currently, carrier particles are most often sized between 50 and 150 microns and therefore have poor flow properties. Poor flow properties lead to variability between doses from dry powder inhalers. According to various aspects of the disclosure, large carrier particles, for example, greater than 500 microns or greater than 1000 microns or from greater than 5000 microns, can overcome poor flow because their sizes are much greater, thereby leading to improved dose uniformity.

Conventional carrier particles have been comprised of mainly lactose, sucrose, glucose, and mannitol. Studies are currently being performed to evaluate the suitability of different sugars. So far, only lactose is the only acceptable carrier for dry powder aerosols in the USA. This is because carrier particles included in inhalers are typically expelled from the inhaler device when the patient aerosolizes the dose. These conventional carrier particles are thus entrained in the patients' inhalation air flow streams and the particles generally deposit in the mouth, throat, and airways. Therefore, the conventional carrier particles must be made of relatively inert materials such as, for example, sugars.

In accordance with various aspects of the disclosure, the large carrier particles, for example, greater than 500 microns or greater than 1000 microns, are not restricted by material selection because the larger particle sizes do not enter the lungs of the patient. In addition, because of the larger size carrier particles, retaining mechanisms 130 can be readily employed in inhalation devices in accordance with the disclosure to capture the large carrier particles within the inhalation device. For example, screens, meshes, filters, channels, orifices, nozzles, etc. can be used in such inhalation devices, whereby the openings 132 are smaller than the large carrier particle size but larger than the drug particle size. Therefore, the large carrier particles are retained in the inhalation device. It also possible, because of the large carrier particle size, for example, greater than 500 microns or greater than 1000 microns or greater than 5000 microns, to capture the large carrier particles using other methods such as aerodynamic sorting and separation of the carrier particles or magnetic capture of the carriers.

According to various aspects of the disclosure, the large carrier particles may comprise biodegradable and/or biocompatible materials because the large carrier particles, for example, greater than 500 microns or greater than 1000 microns, are more easily captured by inhalation devices and are not intended to be inhaled. Any known material can be used. For example, according to various aspects, the carrier particles may comprise sucrose, polystyrene, PTFE, silicone glass, or silica gel or glass.

In accordance with various aspects, working agents such as therapeutic agents for use with the large carrier particles according to the disclosure may include drugs for the treatment of lung diseases and/or systemic diseases. Drugs for systemic diseases may require absorption into the blood stream. According to various aspects, therapeutic agents may include micronized drugs (less than 10 microns, greater than 0.5 microns) and/or nanoparticle drugs (less than 500 nanometers). To improve performance of formulation characteristics such as flow, blending, adhesion to carrier particles, etc., drugs can be blended with other excipients such as leucine, magnesium stearate, fine sugar particles, or the like. It should be appreciated that a formulation according to the disclosure may include two or more drugs. For example, in some aspects, a beta agonist and corticosteroid drug can be blended with the large carrier particles either together or separately and then both placed in the inhaler for delivery of the two drugs to the lungs during inhalation by the patient.

Blending of drug with the large carrier particles in accordance with the disclosure may be achieved by typical methods such as, for example, v-shell mixers, turbula mixers, and other mixers. The large carrier particles can be blended to uniformity with the drug particles. The mixing of drug with the large carrier particles may be optimized by selecting appropriate mixing times. Selection of surface properties of the large carrier particles may also be modified to enhance the blending and uniform mixing of the drug with the carrier.

Blend uniformity may be monitored using experiments that sample the mixture periodically during blending. Uniformity should result in coefficients of variation between samples within the mixture of less than 10-15%.

Powder flow of dry powder formulations may be improved by increasing the particle size of the carrier particles. For example, it has been demonstrated that powder flow properties deteriorate nearly exponentially with decreasing particle size by Hou and Sun (Abstract presented at American Association of Pharmaceutical Sciences Annual Meeting, 2007, San Diego). For a powder exhibiting marginal flow properties during powder handling, particle or bead size enlargement may be an effective means to improve flow properties and manufacturability. To obtain substantially constant powder flow of a given formulation, granule/particle size should be carefully controlled. Flow properties of powders constituted of larger particles are less sensitive to variations in external stress such as those experienced during scale up activities.

According to various aspects of the disclosure, powder flow may be controlled using particle size, density, and particle shape of the large carrier particles, for example, greater than 500 microns or greater than 1000 microns or greater than 5000 microns.

Packaging of the carrier particle system can be achieved by using conventional methods of loading dry powder inhaler formulations into the inhaler such as blister strip packaging, packaging in capsules for insertion into the device, packaging into device reservoirs, and other methods generally used and known.

According to various aspects, large carrier particles consistent with the disclosure, for example, greater than 500 microns or greater than 1000 microns, or greater than 5000 microns, can be used in commercially available devices on the market today (for example the Aerolizer™ marketed by Schering Plough). Development of novel devices that retain carrier particles using screens, meshes, filters, and other separation methods is ongoing, and such devices can also be used. Devices that allow release of carrier particles can also be used. In may be desirable to use devices that maximization of forces that cause detachment using optimized structures within the device. For example, causing the carrier particles to impact once or repeatedly on a mesh during inhalation by the patient for the significant part of the inhalation effort may be desirable.

Performance of the carrier particle systems including large carrier particles consistent with the disclosure, for example, greater than 500 microns or greater than 1000 microns or greater than 5000 microns, may be monitored, for example, via blend uniformity studies, emitted dose studies, powder flow characterization, aerosol dispersion studies, cascade impaction studies relevant for lung deposition predictions, fine particle fraction, fine particle dose, respirable fraction, emitted dose, throat deposition, mass median aerodynamic diameter, effect of time and use on the stability and variability of the formulation.

According to some aspects, the large carrier particles, for example, greater than 500 microns or greater than 1000 microns or greater than 5000 microns, can also be used for delivery of therapeutic agents to the nasal cavity. The large carrier particles may have one or more of the above mentioned advantages of improved efficiency, better powder flow, better uniformity, flow rate independence, etc. In addition, because intranasal delivery of excipient may be reduced or eliminated in accordance with the disclosure, irritation to the nasal mucosa can be avoided. This may be desirable for minimizing mucus production, sneeze reflex, and/or particle clearance from the nasal cavity.

The invention will now be illustrated in further detail by the following non-limiting examples.

### Example 1

To demonstrate the applicability and usefulness of the present invention in dry powder inhaler formulations for pulmonary drug delivery, standard lactose/budesonide dry powder formulations were compared with novel formulations comprised of large (3.38-4.38 millimeter diameter size range) polystyrene carrier particles blended with lactose.

A 2% budesonide in lactose blend (63-90 micrometer diameter size range) was prepared by geometric dilution of 20 mg of micronized budesonide with 980 mg of lactose monohydrate. This mixture was blended with a Turbula™ mixer for 40 minutes. To ensure the homogenous mixing of the lactose and budesonide, a blend uniformity test was performed by sampling the powder from four random areas of the vial containing the sample. The results reveal that the blend was uniform. Approximately 20 mg of the lactose/budesonide blend were loaded into gelatin capsules, which were placed into an Aerosolizer™ dry powder inhaler and passed through a next generation cascade impactor (NGI™) with a flow rate of 60 L/min for a period of four seconds. For the novel carrier particle formulations, 21.6 mg of micronized budesonide was added to a vial containing 85.2 mg of spherical polystyrene beads (3.38-4.38 millimeter diameter size range; density=) and mixed manually with a spatula for one minute. Four polystyrene beads were selected for each run, placed into an Aerosolizer™ dry powder inhaler and passed through a next generation cascade impactor (NGI™) with a flow rate of 60 L/min for a period of four seconds Both the standard lactose/budesonide formulations and the polystyrene bead/budesonide formulations were each run through the NGI four times. The drug remaining in the capsule or on the beads was collected, along with drug deposited from in the inhaler, throat, pre-separator, stage 1 (>5 micrometers) and stages 2-7 (corresponding to diameters<5 micrometers, or the fine particles at 60 L/min) and analyzed. The amount of drug deposited in the throat, and the fine particle fraction for each formulation are summarized below:

| Formulation | Throat Deposition | Fine Particle Fraction |
|---|---|---|
| Lactose/Budesonide-1 | 66.40% | 18.92% |
| Lactose/Budesonide-2 | 56.30% | 20.99% |
| Lactose/Budesonide-3 | 51.58% | 19.24% |
| Lactose/Budesonide-4 | 54.59% | 16.05% |
| Polystyrene/Budesonide-1 | 18.26% | 60.71% |
| Polystyrene/Budesonide-2 | 20.19% | 58.86% |
| Polystyrene/Budesonide-3 | 8.44% | 70.57% |
| Polystyrene/Budesonide-4 | 12.08% | 64.96% |

The graph below depicts the averages of the fraction of the emitted dose collected from the throat, and the fine particle fraction with the error bars corresponding to ±1 standard deviation. As can be readily seen, the throat deposition and fine particle fraction are approximately opposites of each other between the standard lactose/budesonide formulations and the novel polystyrene/budesonide formulations, demonstrating the superiority of the large polystyrene particles when compared to the standard dry powder formulation.

Thus, apart from the enhanced fine particle fraction (18.6% lactose formulation versus 63.8% polystyrene formulation) achieved with the novel carrier particles, there is a significant decrease in the amount of drug that deposits in the throat (57.1% lactose formulation compared to 14.7% polystyrene formulation), thereby minimizing potentially adverse side-effects.

### Example 2

To determine the usefulness of dry powder formulations composed of novel carrier particles for use under conditions where the inhalation flow rate is reduced compared to a healthy patient, such as would be found in patients with pulmonary disorders, the in vitro lung deposition studies of the novel dry powder formulations were performed at 30 L/min (as compared to 60 L/min in Example 1) against standard lactose/budesonide dry powder formulations. 20 mg of the 2% budesonide blend described in Example 1 were loaded into gelatin capsules, placed into an Aerosolizer™ dry powder inhaler and passed through a next generation cascade impactor (NGI™) with a flow rate of 30 L/min for a period of four seconds. Four polystyrene beads taken from the blend described in Example 1 were used for each dry powder formulation, placed into an Aerosolizer™ dry powder inhaler and passed through a next generation cascade impactor (NGI™) with a flow rate of 30 L/min for a period of four seconds. Both the standard lactose/budesonide formulations and the polystyrene bead/budesonide formulations were each run through the NGI three times. The drug remaining in the capsule or on the beads was collected, along with drug deposited from in the inhaler, throat, pre-separator, stage 1, stage 2, and stages 3-7 (corresponding to diameters<5 micrometers, or the fine particles at 30 L/min) and analyzed. The amount of drug deposited in the throat, and the fine particle fraction for each formulation are summarized below:

| Formulation | Throat Deposition | Fine Particle Fraction |
|---|---|---|
| Lactose/Budesonide-1 | 54.36% | 7.67% |
| Lactose/Budesonide-2 | 52.92% | 10.00% |
| Lactose/Budesonide-3 | 52.47% | 7.28% |
| Polystyrene/Budesonide-1 | 3.17% | 51.97% |
| Polystyrene/Budesonide-2 | 5.16% | 46.74% |
| Polystyrene/Budesonide-3 | 7.28% | 36.93% |

The graph below depicts the averages of the fraction of the emitted dose collected from the throat, and the fine particle fraction with the error bars corresponding to ±1 standard deviation. Similar to Example 1, the novel large polystyrene carrier particles significantly outperform the lactose formulations with regards to both minimized throat deposition (53.2% lactose formulation versus 5.2% polystyrene formulation) and enhanced fine particle fraction (8.32% lactose formulation versus 45.2% polystyrene formulation).

Furthermore, while the average fine particle fraction of the lactose formulations at 30 L/min was less than half (44.7%) what it was at 60 L/min (18.6% compared to 8.32%), the average fine particle fraction obtained from the polystyrene formulations at 30 L/min was 71% of the fine particle fraction at 60 L/min (45.2% compared to 63.8%), demonstrating that the fine particle fraction of the novel large carrier particles is more resilient against changes in inspiratory flow rate.

### Example 3

To determine the effect of altering the size range of the novel polystyrene carrier particles, in vitro drug deposition studies were performed using three different size ranges of polystyrene beads (4.38-5.38 mm (large), 3.38-4.38 mm (medium), and 1.44-2.36 mm (small)). Polystyrene bead/budesonide blends were prepared for each of the preceding size ranges as described in Example 1. Polystyrene beads taken from the blends were placed into an Aerosolizer™ dry powder inhaler and passed through a next generation cascade impactor (NGI™) with a flow rate of 60 L/min for a period of four seconds. Each polystyrene bead/budesonide size formulation was run through the NGI three times. The drug remaining on the beads was collected, along with drug deposited from in the inhaler, throat, pre-separator, stage 1, and stages 2-7 (corresponding to diameters<5 micrometers, or the fine particles at 60 L/min) and analyzed. The amount of drug deposited in the throat, and the fine particle fraction for each formulation are summarized below:

| Run | FPF | Throat |
|---|---|---|
| Large #1 | 67.36% | 15.23% |
| Large #2 | 61.13% | 12.03% |
| Large #3 | 63.89% | 13.39% |
| Medium #1 | 68.04% | 10.82% |
| Medium #2 | 65.86% | 13.79% |
| Medium #3 | 63.33% | 17.6% |
| Small-1 | 54.65% | 14.18% |
| Small #2 | 65.51% | 12.62% |
| Small #3 | 63.64% | 14.15% |

The averages of the three runs are shown in the graph below (the error bars corresponding to ±1 standard deviation), and indicated no significant differences between the large (4.38-5.38 mm), medium (3.38-4.38 mm) and small (1.44-2.36 mm) polystyrene beads in terms of both fine particle fraction (64.1%, 65.7% and 61.2% respectively) and throat deposition (13.5%, 14.1% and 13.6% respectively). However, when compared to the standard lactose/budesonide blends, the fraction deposited on the throat remains significantly smaller, while fine particle fraction is significantly greater, for all three size ranges investigated.

### Example 4

Five different size ranges of polystyrene (average density=0.0242 g/cm³) were blended with micronized budesonide (Spectrum Chemicals) and investigated as carrier particles. The size ranges and masses of the beads and budesonide are shown below:

Polystyrene/Budesonide Formulations

| Size Range | Bead Mass (mg) | Budesonide Mass (mg) |
|---|---|---|
| 841-1168 um | 59.7 | 25.2 |
| 1168-1411 um | 67.6 | 29.7 |
| 1411-2360 um | 57.8 | 30.2 |
| 3380-4380 um | 65.2 | 24.0 |
| 4380-5380 um | 42.3 | 20.3 |

The drug and beads were blended together in aluminum vials for 10 minutes with a Turbula orbital mixer. The formulations were stored in a dessicator until used.

Silica gel (density=1.83 g/c^{m3}) carrier particles/micronized budesonide formulations were made using three different size ranges of silica gel beads: 600-841 micrometers, 841-1168 micrometers, and 1168-1411 micrometers. The masses of the beads and budesonide used in each size range formulation are shown in the table below:

Silica gel/Budesonide Formulations

| Size Range | Bead Mass (mg) | Budesonide Mass (mg) |
|---|---|---|
| 600-841 um | 863.2 | 29.5 |
| 841-1168 um | 1115.6 | 29.7 |
| 1168-1411 um | 1450.4 | 29.1 |

The drug and beads were blended together in aluminum vials for 10 minutes with a Turbula orbital mixer. The formulations were stored in a dessicator until used.

A single size range (841-1168 micrometers) of glass beads (mass=1.631 grams; density=2.48 g/cm³) were mixed with 33.9 mg of micronized budesonide in an aluminum vial for 10 minutes with a Turbula orbital mixer. The formulation was stored in a dessicator until used.

Three size ranges of sucrose beads (density=1.54 g/cm³) were blended with budesonide and examined as carrier particles. The size ranges and masses of beads and drug used in each formulation are shown below:

| Sucrose/Budesonide Formulations | | |
|---|---|---|
| Size Range | Bead Mass (mg) | Budesonide Mass (mg) |
| 841-1168 um | 837.8 | 22.7 |
| 1168-1141 um | 638.9 | 20.5 |
| 1411-2867 um | 781.5 | 20.5 |

The drug and beads were blended together in aluminum vials for 10 minutes with a Turbula orbital mixer. The formulations were stored in a dessicator until used.

The figure below shows the fine particle fractions and throat depositions for the polystyrene, glass, silica gel, and sucrose formulations.

### Example 5

### Bead Carrier Particles

Carrier particles, comprised of low density (<0.300 g/cm³) polystyrene beads, with geometric diameter between 4.35 and 5.35 microns were placed into a glass vial (25 mL volume capacity) with micronized budesonide (d₉₀<5 microns) as the active pharmaceutical ingredient. 1 polystyrene bead was placed into a vial in addition to 1 milligram of budesonide powder. The amount of drug loaded onto a single polystyrene carrier particle ranged from 360-480 micrograms, comparable to the 400 micrograms loaded in a standard 20 mg dose of 2% (w/w) drug/lactose carrier formulation. A single budesonide-coated polystyrene bead was placed into the capsule chamber of an Aerolizer dry powder inhaler, which was connected to a Next Generation Cascade Impactor. In vitro drug dispersion studies were performed at a volumetric flow rate of 60 L/min for 4 seconds. The budesonide remaining on the polystyrene carrier, or depositing on the inhaler, throat, pre-separator, and stages 1-8 of the cascade impactor was collected and quantified.

The respirable fraction (the fraction of the total dose that deposits in the deep lung) for the polystyrene carrier particles ranged between 45 and 50%. The respirable fraction from standard lactose carrier particles is generally below 25%. As a result, the large-size polystyrene carrier particles in accordance with the disclosure may reduce cost by reducing the amount of working agent, for example, drug or therapeutic agent, that must be deposited on the carrier particles in order to deliver a sufficient amount of the working agent to the airway of a patient. In addition, the large-size polystyrene carrier particles in accordance with the disclosure may deposit less working agent in the throat and mouth of a patient, thus reducing potential side effects to the patient.

### Example 6

### Flake Carrier Particles

Carrier particles were prepared in the following method. Flake-shaped carrier particles between 1 and 3 millimeters in length, 1 and 3 millimeters in width, 100 microns in thickness and composed of hydroxypropyl methylcellulose (HPMC) were obtained by fragmenting a HPMC two-piece capsule. The general shape of the resulting capsule fragments were of irregular quadrilaterals, fitting the above dimensions, although a more accurate description would be that they were polygons with non-uniform sides (both in length and number), and angles. 32.4 milligrams of HPMC carrier particles (the collective fragments of 1 piece of the original 2 piece capsule, capsule size 1) were placed into a glass vial (25 mL volume capacity). Added to this was 2 milligrams of micronized budesonide powder (primary particle size=d₉₀<5 microns, where d₉₀ is the volume diameter of 90% of the particles) as the active pharmaceutical ingredient. In a one-off trial, the amount of drug loaded on the HPMC particles was 1.235 milligrams. Standard dry powder formulations with lactose carrier particles (<90 micron diameter) generally load 400 micrograms (0.400 milligrams) of drug. The budesonide-coated HPMC fragments were placed into the capsule chamber of an Aerolizer dry powder inhaler, which was connected to a Next Generation Cascade Impactor. In vitro drug dispersion studies were performed at a volumetric flow rate of 60 L/min for 4 seconds. The budesonide remaining on the HPMC carriers, or depositing on the inhaler, throat, pre-separator, and stages 1-8 of the cascade impactor was collected and quantified.

The fine particle fraction (the percent of the dose emitted from the inhaler that deposits in the deep lung) was 78%, compared to less than 30% for standard lactose carrier particles. This example illustrates that the shape of the carrier particle is not restricted to spherical beads. The mechanism of action describes a carrier particle that is retained within the dry powder inhaler device during inhalation, allowing for a wide range of materials, sizes and morphologies to be employed as drug carriers in dry powder formulations.

It is noted that, as used in this specification and the appended claims, the singular forms “a,” “an,” and “the,” include plural referents unless expressly and unequivocally limited to one referent. Thus, for example, reference to “a particle” may include two or more different particles. As used herein, the term “include” and its grammatical variants are intended to be non-limiting, such that recitation of items in a list is not to the exclusion of other like items that can be substituted or other items that can be added to the listed items.

It will be apparent to those skilled in the art that various modifications and variations can be made to the formulations, carrier particles, inhalers, and methods of the present disclosure without departing from the scope of the invention. Other embodiments of the invention will be apparent to those skilled in the art from consideration of the specification and practice of the invention disclosed herein. It is intended that the specification and examples be considered as exemplary only.

1. A dry powder inhaler comprising:
a drug chamber configured to contain a formulation including carrier particles and working agent particles;
a mouthpiece configured to direct flow of working agent particles to a user; and
a retaining member proximal the mouthpiece, the retaining member be sized and arranged to prevent flow of substantially all carrier particles to the user while permitting flow of working agent particles to a user.

2. The inhaler of claim 1, wherein the retaining member is configured to prevent flow of carrier particles having a sieve diameter greater than about 250 microns while permitting flow of working agent particles having a sieve diameter less than about 250 microns.

3. The inhaler of claim 1, wherein the retaining member is configured to prevent flow of carrier particles having a sieve diameter greater than about 500 microns while permitting flow of working agent particles having a sieve diameter less than about 500 microns.

4. The inhaler of claim 1, further comprising a formulation including carrier particles for delivering working agent to the pulmonary system of a patient via a dry powder inhaler, the carrier particles having an average sieve diameter greater than about 500 μm.

5. The inhaler of claim 1, further comprising a formulation including carrier particles for delivering working agent to the pulmonary system of a patient via a dry powder inhaler, the carrier particles having an average sieve diameter greater than about 1000 μm.

6. The inhaler of claim 1, further comprising a formulation including carrier particles for delivering working agent to the pulmonary system of a patient via a dry powder inhaler, the carrier particles having an average sieve diameter of greater than about 5000 μm.

7. The inhaler of claim 4, wherein the formulation further comprises particles of working agent adhered to the carrier particles.

8. The inhaler of claim 4, wherein the carrier particles comprise one of polystyrene, PTFE, silicone glass, silica gel, and silica glass.

9. The inhaler of claim 4, wherein the carrier particles comprise biodegradable material.

10. The inhaler of claim 9, wherein the carrier particles comprise sucrose.

11. A formulation for a dry powder inhaler, the formulation comprising carrier particles for delivering working agent to the pulmonary system of a patient via a dry powder inhaler, the carrier particles comprising one of polystyrene, PTFE, silicone glass, silica gel, and silica glass, the carrier particles having an average sieve diameter greater than about 500 μm.

12. The formulation of claim 11, further comprising particles of working agent adhered to the carrier particles.

13. The formulation of claim 11, wherein the carrier particles have an average sieve diameter greater than about 1000 μm.

14. The formulation of claim 11, wherein the carrier particles have an average sieve diameter of greater than about 5000 μm.

15. A formulation for a dry powder inhaler, the formulation comprising carrier particles for delivering working agent to the pulmonary system of a patient via a dry powder inhaler, the carrier particles having an average sieve diameter greater than about 1000 μm.

16. The formulation of claim 15, further comprising particles of working agent adhered to the carrier particles.

17. The formulation of claim 15, wherein the carrier particles have an average sieve diameter of greater than about 5000 μm.

18. The formulation of claim 15, wherein the carrier particles comprise biodegradable material.

19. The formulation of claim 15, wherein the carrier particles comprise nonbiodegradable material.

20. The formulation of claim 19, wherein the carrier particles comprise one of polystyrene, PFTE, silicone glass, silica gel, and silica glass.
